(19) United States
(12) Patent Application Publication
(10) Pub. No.: US 2008/0194998 A1
(43) Pub. Date: Aug. 14, 2008

(54) Method, Device and Computer-Readable Medium for Evaluating Prevalence of Different Patient Postures

(76) Inventors: Nils Holmstrom, Jarfalla (SE); Malin Ohlander, Stockholm (SE)

Correspondence Address: SCHIFF HARDIN, LLP;PATENT DEPARTMENT, 6600 SEARS TOWER, CHICAGO, IL, 60606-6473, US

(21) Appl. No.: 11/913,785

(22) PCT Filed: May 24, 2005
(86) PCT No.: PCT/SE05/00777

§ 371 Date: Nov. 7, 2007

(51) Int. Cl. A61B 5/00

(52) U.S. Cl. 600/595

(57) ABSTRACT

In a method, device and compute-readable medium for evaluating the prevalence of different postures of a patient, signals are sensed that indicate the posture of the patient during a monitoring period having a predetermined length, specific body postures of the patient are determined during this monitoring period from the sensed signals, the amount of time the patient spends in each of the specific postures is measured, information regarding each specific posture, and the associated amount of time spent in that posture, are stored, and the prevalence of the different postures of the patient is evaluated by classifying the stored information with respect to specific postures and the amount of time in each posture position.

### TECHNICAL FIELD

The present invention generally relates to implantable medical devices, such as cardiac pacemakers and implantable cardioverter/defibrillators, and in particular to a method and medical device for evaluating the prevalence of different postures of a patient.

### BACKGROUND OF THE INVENTION

A severe problem associated with measurement of, inter alia, the blood pressure of the chambers, contractility, endocardial acceleration, blood flow, coronary blood flow, the sinus rate, the electrical bio-impedance, such as the thoracic impedance and the cardiogenic impedance is that the accurateness and reliability, and, hence the repeatability, of the obtained signals are greatly affected by factors like the body position of the patient, patient activity levels, heart rate, etc. For example, it has been found that the body position of the patient is of major importance with regard to the blood pressure of the chambers, contractility, endocardial acceleration, blood flow, coronary blood flow, the sinus rate, the electrical bio-impedance, such as the thoracic impedance and the cardiogenic impedance, etc.

Repeatable measurements of such parameters are of a great value for identifying changes of many different conditions in the body of a patient. For example, electrical bio-impedance signals has been found to be an effective measure for identifying changes of many different conditions in the body of a patient, such as incipient pulmonary edema and the progression of pulmonary edema due to CHF, i.e. the accumulation of fluids in the lung-region associated with pulmonary edema affects the thoracic impedance, or more specifically the DC impedance level, since the resistivity of the lung changes in accordance with a change of the ratio of fluid to air. In addition to the thoracic impedance, the cardiogenic impedance, which is defined as the impedance or resistance variation that origins from cardiac contractions measured by electrodes inside or on the surface of the body, can be used for identifying changes of different conditions in the heart of a patient. For example, parameters such as the systolic and diastolic slopes, pre-ejection period and left ventricular ejection time indicative of different functions of the heart can be extracted from the cardiogenic impedance. The cardiogenic impedance variation correlates to the volume changes of the heart chambers, which can be used as an indication of the dynamic blood filling. Hence, changes of these parameters due to a change in the heart, for example, caused by a disease such as heart failure can be detected by monitoring or detecting changes of the cardiogenic impedance.

Furthermore, parameters associated with the heart, such as blood pressure of the chambers, contractility, endocardial acceleration, blood flow, coronary blood flow, the sinus rate etc., is very useful for diagnostic and/or therapeutic purposes, e.g. for identifying changes of different conditions in the heart of a patient. In order to be able to monitor changes of conditions of a patient, the measurements of the parameters, such blood pressure of the chambers, contractility, endocardial acceleration, blood flow, coronary blood flow, the electrical bio-impedance, such as the thoracic impedance and the cardiogenic impedance, must be substantially repeatable.

A number of attempts to eliminate or filter out error sources such as the body position of the patient, patient activity levels, heart rate frequency, etc have therefore been proposed. For example, U.S. Pat. No. 6,104,949 discloses a method and device for treatment of CHF, in which changes in the posture of the patient is correlated with changes of the trans-thoracic impedance. A posture sensing means indicates whether the patient lies down or is standing and the measurement of the trans-thoracic impedance is then correlated with periods when the patient is lying down. Thus, according to this known method, the obtaining of the impedance signals are correlated with periods when the patient is lying down.

However, it has recently been found that the posture or position dependence also is of a significant magnitude regarding different positions even when the patient is lying down, for example, whether the patient is lying on a side or is lying on the back. For example, regarding impedance measurements, a major reason is that the measurement depends on the measurement vector, i.e. the vector between the nodes that the current is applied between and the vector the voltage is measured between. When the body shifts position, these vectors will change since the gravity will influence, for example, tissue between the nodes and how it moves. Tests performed on animals have shown that the trans thoracic impedance may vary up to 20% depending on which position the animal was lying in. Furthermore, in many applications there may also be desirable or even necessary to perform the measurements regularly at the same conditions and the time period.

Accordingly, there is a need of a method and medical device that are capable of evaluating the prevalence of different postures of a patient, which evaluation can be used to identify the most suitable posture for performing measurements in order to obtain measurements with a high degree of repeatability and accurateness.

### BRIEF DESCRIPTION OF THE INVENTION

Thus, an object of the present invention is to provide a method and medical device that are able to evaluate the prevalence of different postures of a patient.

Another object of the present invention is to provide a method and medical device that are able to obtain repeatable and accurate signals indicative of at least one physiological parameter.

These and other objects are achieved according to the present invention by providing a method, medical devices, and a computer readable medium having the features defined in the independent claim. Preferable embodiments of the invention are characterised by the dependent claims.

In the context of this application, the term “impedance” refers to the DC component of the impedance. The measured impedance consists of a DC component and an AC component, where the DC component is the baseline around which the AC component fluctuates. The DC component reflects the amount of tissue and fluids that are located between the measuring points that the impedance is measured in-between and the AC components reflects how respiration and cardiac activity influence the impedance signal.

For the purpose of clarity, the term “intra thoracic impedance” refers to an impedance measurement over the thorax by using an implantable medical device, i.e. an impedance measurement where the impedance measurement vector spans over the thorax.

Moreover, in order to clarify, the term “cardiogenic impedance” is defined as the impedance or resistance variation that origins from cardiac contractions or, in other words, the cardiac component of the impedance measured between electrodes in contact with the body.

According to an aspect of the present invention, there is provided a method for evaluating the prevalence of different postures of a patient comprising the steps of: sensing signals indicating the posture of the patient during a monitoring period having a predetermined length; determining specific body postures of the patient during the monitoring period using the signals; measuring the amount of time the patient spends in each of the specific postures; storing information regarding each specific posture and the amount of time spent in each posture; and evaluating the prevalence of the different postures of the patient by classifying the stored information with respect of specific postures and the amount of time spent in corresponding postures.

According to a second aspect of the present invention, there is provided a medical device for evaluating the prevalence of different postures of a patient comprising: a position sensing means arranged to sense the posture of the patient; determining means arranged to determine specific body postures of the patient during a monitoring period having a predetermined length using position signals from the position sensing means; timing means arranged to measure the amount of time the patient spends in each of the specific postures; storing means arranged to store information regarding each specific posture and the amount of time spent in each posture; and evaluating means arranged to evaluate the prevalence of the different postures of the patient by classifying the stored information with respect of specific postures and the amount of time spent in corresponding postures.

According to a third aspect of the present invention, there is provided a computer readable medium comprising instructions for bringing a computer to perform a method according to the first aspect.

Thus, the invention is based on the idea of recording patient posture signals during a predetermined patient monitoring period and using the history of the posture signals to determine the prevalence of the different postures of the patient.

This solution provides several advantages over the existing solutions. One advantage is that information regarding the posture pattern of a specific patient can be collected in an efficient and reliable way, which information, for example, may provide basis for a selection of specific posture as a triggering event for a measurement session of a posture sensitive physiological parameter.

According to a preferred embodiment of the present invention, at least one measurement criterion is selected in dependence of the evaluation. For example, a set of specific signals suitable for a specific diagnostic purpose can be selected as measurement criterions, e.g. a specific position occurring at regular intervals. Accordingly, it is possible to customize the measurement criterions for a specific patient and/or for a measurement of a specific parameter, such as blood pressure (P), blood pressure variation (dP/dt), heart sound, contractility, endocardial acceleration, blood flow, coronary blood flow, electrical bio-impedance, such as the cardiogenic impedance or the intra thoracic impedance, etc.

In another embodiment of the present invention, a measurement session in order to measure at least one physiological parameter is initiated when the selected at least one measurement criterion is satisfied. By performing the measurement only when the measurement criterion is satisfied, for example when the patient is in a specific posture, substantially repeatable signals can be obtained. Thereby, it is possible, for example, to monitor or detect changes of a condition of the patient or trends in the development of a condition of a patient in an effective way. Furthermore, this also entails that variations in the signals due to measurements in different body positions can be substantially eliminated, which is an evident risk with the method disclosed in, for example, U.S. Pat. No. 6,104,949, where the impedance measurements is correlated with moments when the patient is lying down and, therefore, the measurements are, in practical, performed in a number of different positions, i.e. when the patient is lying on either side or when the patient is lying on the back, etc. An additional advantage is that the measurements are initiated only when the predetermined measurement criterion is satisfied whereby a high efficiency with respect to current consumption is achieved.

According to another embodiment of the present invention, an activity level of the patient during patient monitoring period, it is determined whether the activity level is within at least one predetermined range, the information regarding the activity level range of the patient is stored together with each specific posture and the amount of time spent in each posture, and using the information regarding the activity level range of the patient in the evaluation by classifying the information with respect of specific postures, the amount of time spent in corresponding postures, and activity level range. Thereby, even more reliable measurements can be obtained since also the activity level of the patient is used to provide basis for a selection of a set of signals for the initiating of a measurement session.

In one embodiment, the measurements are initiated in order to sense the intra thoracic impedance. Thereby, the progression of pulmonary edema can be monitored since the accumulation of fluids in the lung-region associated with pulmonary edema affects the thoracic impedance, or more specifically the DC impedance level, since the resistivity of the lung changes in accordance with a change of the ratio of fluid to air. The DC impedance level is negatively correlated with the amount of fluids in the lung. Due to the fact that pulmonary edema is a symptom of CHF, the development of CHF can be monitored indirectly by means of the intra thoracic impedance. For example, studies have shown that hospitalization due to the development of acute CHF with the symptom pulmonary edema was preceded two or three weeks by a drop in the DC impedance by approximately 10-15%.

Further objects and advantages of the present invention will be discussed below by means of exemplifying embodiments.

### BRIEF DESCRIPTION OF THE DRAWINGS

In the following detailed description, reference will be made to the accompanying drawings, of which:

FIG. 1 is schematic diagram showing a medical device implanted in a patient in which device the present invention can be implemented.

FIG. 2 is block diagram of the primary functional components of a first embodiment of the medical device according to the present invention.

FIG. 3 is block diagram of the primary functional components of a second embodiment of the medical device according to the present invention.

FIG. 4 is a flow chart illustrating the steps in accordance with one embodiment of the present invention for evaluating the prevalence of different postures of a patient.

FIGS. 5a and 5B show, during an eight hour period, the different positions of a patient and the amount of time the patient spends in each position and the corresponding position histogram for the information shown in FIG. 5a, respectively.

FIG. 6 is a flow chart illustrating the steps in accordance with one embodiment of the present invention for initiating a measurement session in order to measure a physiological parameter of a patient using the evaluation of the prevalence of the different postures of the patient.

### DETAILED DESCRIPTION OF THE INVENTION

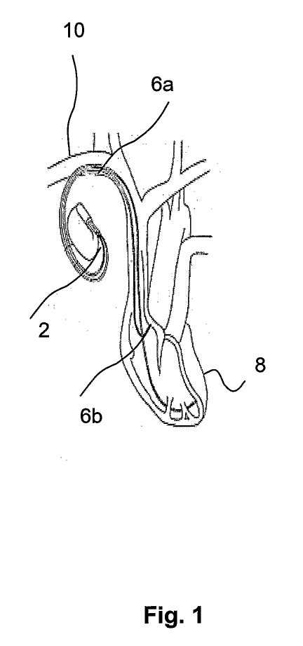

With reference to FIG. 1 there is shown a schematic diagram of a medical device implanted in a patient in which device the present invention can be implemented. As seen, this embodiment of the present invention is shown in the context of a pacemaker 2 implanted in a patient (not shown). The pacemaker 2 comprises a housing being hermetically sealed and biological inert. Normally, the housing is conductive and may, thus, serve as an electrode. One or more pacemaker leads, where only two are shown in FIG. 1 namely a ventricular lead 6a and an atrial lead 6b, are electrically coupled to the pacemaker 2 in a conventional manner. The leads 6a, 6b extend into the heart 8 via a vein 10 of the patient. One or more conductive electrodes for receiving electrical cardiac signals and/or for delivering electrical pacing to the heart 8 are arranged near the distal ends of the leads 6a, 6b. As the skilled man in the art realizes, the leads 6a, 6b may be implanted with its distal end located in either the atrium or ventricle of the heart 8.

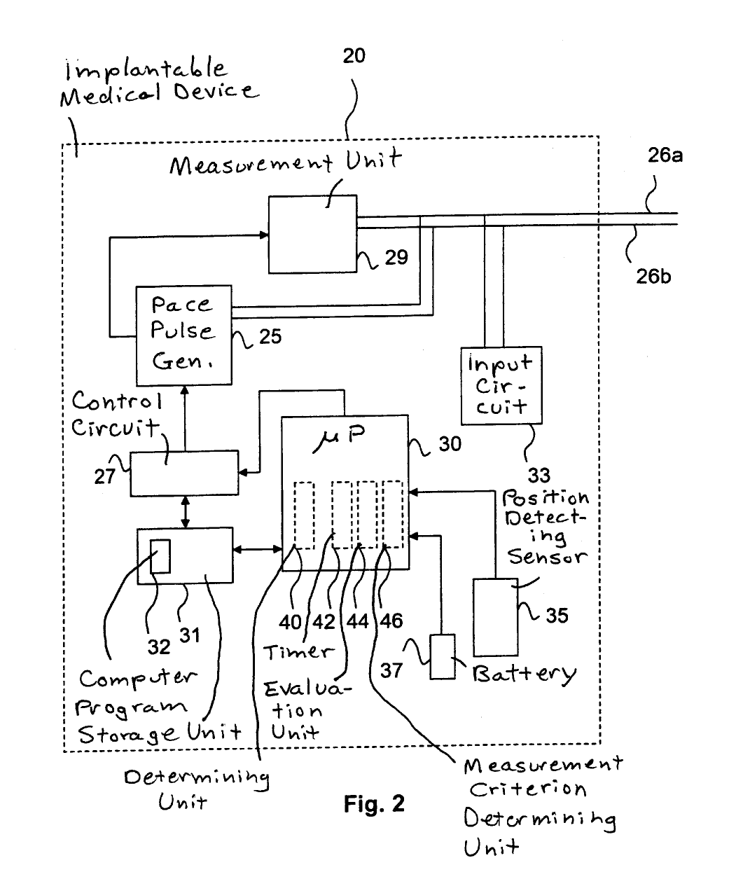

With reference now to FIG. 2, the configuration including the primary components of an embodiment of the present invention will be described. The illustrated embodiment comprises an implantable medical device 20, such as the pacemaker shown in FIG. 1, and leads 26a and 26b, of the same type as the leads 6a and 6b shown in FIG. 1, for delivering signals between the implantable medical device 20 and the patients heart. The leads 26a, 26b may be unipolar or bipolar, and may include any of the passive or active fixation means known in the art for fixation of the lead to the cardiac tissue. As an example, the lead distal tip (not shown) may include a tined tip or a fixation helix. The leads 26a, 26b comprises one or more electrodes (as described with reference to FIG. 1), such a tip electrode or a ring electrode, arranged to, inter alia, transmit pacing pulses for causing depolarization of cardiac tissue adjacent to the electrode(-s) generated by a pace pulse generator 25 under influence of a control circuit 27. The control circuit 27 controls pace pulse parameters such as output voltage and pulse duration.

The control circuit 27 acts under influence of the microprocessor 30. A storing means 31 is connected to the control circuit 27 and the microprocessor 30, which storing means 31 may include a random access memory (RAM) and/or a non-volatile memory such as a read-only memory (ROM). In this embodiment, the storing means 31 comprises a computer program 32 comprising instructions for bringing a computer or a microprocessor to cause method steps in accordance with the present invention. Detected signals from the patients heart are processed in an input circuit 33 and are forwarded to the microprocessor 30 for use in logic timing determination in known manner. The implantable medical device 20 is powered by a battery 37, which supplies electrical power to all electrical active components of the medical device 20. Data contained in the storing means 31 can be transferred to a programmer (not shown) via a programmer interface (not shown) for use in analyzing system conditions, patient information, calculation of surrogate parameters such as systolic and diastolic slopes, the pre-ejection period, or left ventricular ejection time and changing pacing conditions, etc.

Furthermore, the implantable medical device 20 according to the present invention comprises position detecting sensor 35 arranged to detect the body position of a patient. In a preferred embodiment of the present invention, the position detecting sensor is a 3-dimensional orthogonal sensor arranged to sense whether the patient is, for example, standing or in a supine position, a left side position, a right side position, or a prone position. One example of such a 3-dimensional sensor is shown in U.S. Pat. No. 6,044,297. Thereby, the position of the patient can be identified, for example, whether the patient is standing or resting in a supine position, a left side position, a right side position, or a prone position. The position detecting sensor 35 is connected to the microprocessor 30. Furthermore, the device 20 comprises determining means 40 arranged to determine specific body postures of the patient during a predetermined period of time using position signals from the position sensor, timing means 42 arranged to measure the amount of time the patient spends in each specific posture, and evaluating means 44 arranged to evaluate the prevalence of the different postures of the patient by classifying the obtained information with respect of specific postures and the amount of time spent in corresponding postures. The obtained information of the specific postures and amount of time spent in respective posture is stored in the storing means 31.

In a preferred embodiment, the determining means 40, the timing means 42 and the evaluating means 44 are integrated in the microprocessor 30 as being indicated in FIG. 2. The timing means 42 is also arranged to control the length of the monitoring period, i.e. the monitoring period can be set by means of the timing means 42. Moreover, the device comprises measurement criterion determining means 46 connected to the position detecting sensor 35. In this first embodiment, the measurement criterion determining means 46 is incorporated in the microprocessor 30, as indicated in FIG. 2.

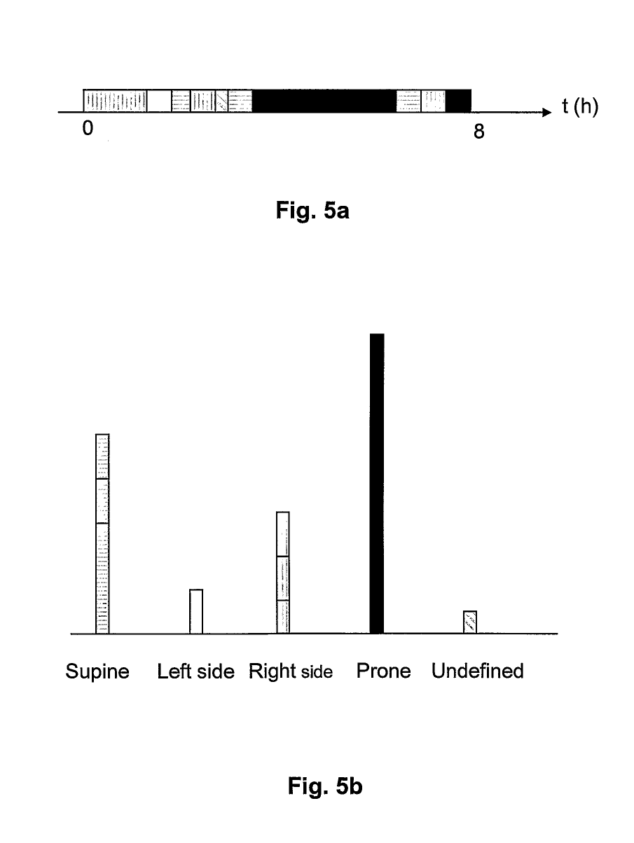

The evaluation means 44 is arranged to evaluate the prevalence of the different postures of the patient by classifying information with respect of specific postures and the amount of time spent in corresponding postures. For example, the evaluation means 44 can be arranged to make histograms showing the amount of time spent in each specific posture, see for example FIGS. 5a and 5b, in accordance with conventional practice within the art. Of course, as the man skilled within the art realizes, other types of statistics can be made. This information can be used as a basis for selecting one or several measurement criterion, as will be discussed in more detail below. The evaluation means may be arranged to select one or several measurement criterions automatically, or the criterions may be selected manually by a doctor or medical attendant by means of the programmer communicating with the medical device 20, for example, via telemetry. Moreover, the resulting statistics, for example, the histogram or histograms can be transferred to the programmer for display on a screen.

The criterion determining means 46 is arranged to determine whether a set of measurement criterion is satisfied, which will be discussed in more detail below. If the set of criterion is satisfied, the criterion determining means 46 is arranged to apply a triggering signal to a measurement means 29, which in a preferred embodiment is an impedance circuit 29 arranged to carry out impedance measurements. The measurement means 29 is arranged to, upon receiving the triggering signal, initiate an measurement session in order to obtain substantially repeatable signals to measure a physiological parameter.

According to this first embodiment, the measurement means 29 is an impedance circuit 29 arranged to carry out impedance measurements. The impedance circuit is arranged to apply excitation current pulses between a first electrode arranged to positioned within a heart of the patient and second electrode in an embodiment where the intra thoracic impedance is measured. The impedance circuit 29 is also arranged to sense the impedance in the tissues between the first and second electrode to the excitation current pulse. Further, the impedance circuit 29 is coupled to the microprocessor 30, where processing of the obtained impedance signals can be performed. In an embodiment where the cardiac component of the electrical bio-impedance is sensed, the impedance circuit 29 is arranged to apply an excitation current pulse between a first electrode and a second electrode arranged to be positioned at different position within the heart of the patient and to sense the impedance in the tissues between the first and second electrode to the excitation current pulse. The microprocessor 30 may be arranged to extract the cardiac component of the sensed impedance. This cardiac component can be used for calculating parameters like systolic and diastolic slopes, the pre-ejection period, or left ventricular ejection time. This calculation can be performed in accordance with conventional practice within the art. The impedance sensing circuit 29 is controlled by the microprocessor 30 and the control circuit 27.

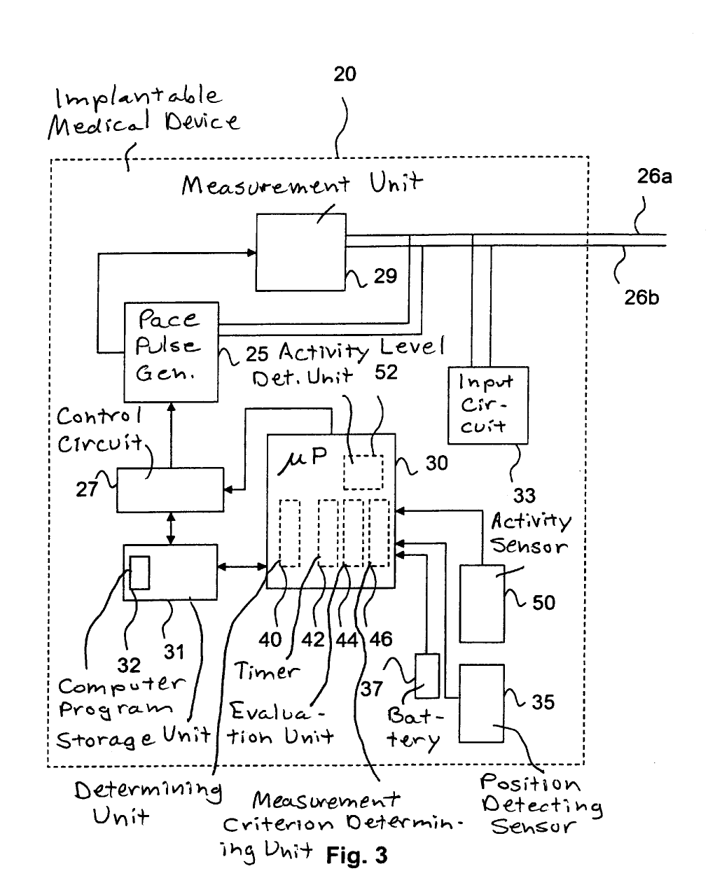

With reference now to FIG. 3, a second embodiment of the present invention will be discussed. Similar parts in the first and second embodiment will be denoted with the same reference numerals. Moreover, the description of like parts and the function of these will be omitted.

According to this embodiment, an activity sensor 50 is incorporated in the medical device in accordance with conventional practice within the art. An activity level determining means 52 is arranged to determine whether the sensed activity level is within at least one predetermined range. In this embodiment, the activity level determining means 52 is incorporated in the microprocessor 30. As will be discussed in further detail hereinafter, one or more activity level ranges may be used as measurement criterion in addition to, for example, the body posture. The storing means 31 is arranged to store information regarding the activity level range of the patient together with each specific posture and the amount of time spent in each posture; and the evaluating means 44 is arranged to evaluate the prevalence of the different postures of the patient by classifying the stored information with respect of specific postures, the amount of time spent in corresponding postures, and the activity level range.

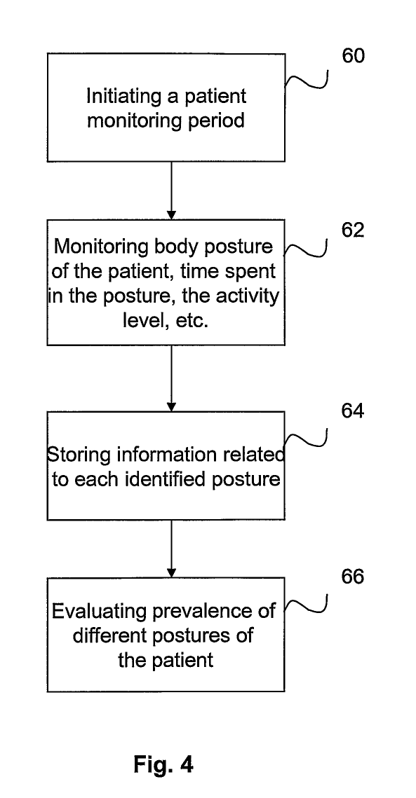

Referring now to FIG. 4, a high-level description of an embodiment of the method according to the present invention will be given. After an implantation of a medical device according to the present invention such as the first or second embodiment discussed above, a patient monitoring period can be executed in order to evaluate the prevalence of different postures of the patient, which evaluation, in turn, can be used to identify the most appropriate combination of signals for performing a sensor measurement session. This monitoring period can be initiated manually by the medical personal using a programmer communicating with the implanted device. The length of the period can be set manually and may last for e.g. 1 to 7 days. In operation, at step 60, the patient monitoring period is initiated in order to obtain information regarding the amount of time the patient spends in different positions at certain sets of criteria. Then, at step 62, the position sensor 35 monitors or detects the positions of the patient in order to detect the body posture of the patient, i.e. the sensor 35 is arranged to supply position indicating signals as described above during the patient monitoring period. The amount of time the patient spends in each position or posture is also measured. At step 64, information regarding each specific posture and the amount of time spent in each posture is stored. In another embodiment of the invention, an activity level sensor senses also the activity level of the patient. It is determined whether the activity level is within at least one predetermined activity level range, the information regarding the activity level range of the patient is stored together with each specific posture and the amount of time spent in each posture. According to a further embodiment of the present invention, also the heart rate of the patient is sensed and it is determined whether the heart rate is within a predetermined range, and the information regarding the heart rate is also stored in the storing means 31.

Thereafter, at step 66, when the patient monitoring period has ended, the prevalence of the different postures of the patient is evaluated by classifying the stored information with respect of specific postures and the amount of time spent in corresponding postures. Alternatively, this evaluation can be performed during the patient monitoring period. In the embodiment comprising an activity level sensor, also the activity level range is used in the evaluation. Furthermore, in the embodiment where the heart rate is sensed, also the heart rate level can be used in the evaluation. According to a preferred embodiment, the stored information is gathered in histograms as can be seen in FIGS. 5a and 5b. FIG. 5a shows, during an eight hour period, the different positions of a patient and the amount of time the patient spends in each position. FIG. 5b shows the corresponding position histogram comprising the information shown in FIG. 5a. The exemplifying results shown in FIGS. 5a and 5b show that the patient during the monitored period of time spends most of the time sleeping on the tummy and least time sleeping on the left side. Moreover, it is shown that the supine position and the right side position are more evenly distributed over the measured period of time in comparison to, for example, prone. In the alternative embodiment of the present invention where the activity level of the patent is sensed, a histogram can be made for each activity level range. For example, one histogram showing the different postures when the patient is in a resting mode and one histogram showing the postures when the patient is in a non-resting mode. Of course, as the skilled man realizes, it is possible to use more than two activity level ranges.

According to the present invention, the above-mentioned evaluation can be used as a base for selecting at least one measurement criterion. The selected measurement criterion can also be memorized in the storing means 31 for use in measurements. That is, once the patient monitoring period is over, the histograms over the patients preferred body, or sensor, positions at different times of the day can be analyzed in order to identify when it is a suitable point of time to measure a specific physiological parameter. There are however a number of conceivable parameters that may be taken into consideration when selecting the at least one measurement criterion:
the specific physiological parameter to be measured;
the total amount of time spent in a specific position;
the number of periods of time spent in a specific position;
the length of the periods of time spent in a specific position;
the frequency of the periods of time spent in a specific position;
the distribution of periods of time in a specific position over the total period of time.

It should be noted that this list is non-exhaustive, and that there are other parameters that also may be used in the selection of measurement criterion. For example, if the activity level of the patient is measured, the above mention parameters may be identified for a specific activity level range or a number of specific activity level ranges, e.g. when the patient is resting, when the patient is walking, and/or when the patient is exercising. This selection of measurement criterions may be performed automatically by the microprocessor in accordance with predefined rules. Such rules may be, for example, that the activity sensor indicates that a low body activity has been detected for a predetermined period, e.g. more than 5 minutes, and the position sensor indicates that the patient is supine. Accordingly, it is, for example, possible to reveal which body positions that are meaningful to use as criterion and which are not. If e.g. the patient never sleeps on the back it would be discovered in the evaluation and thus avoided as a measurement criterion. As an alternative, it is possible to perform the selection of measurement criterions manually by using of the programmer.

As mentioned above, the measurement criterion can be used for triggering a measurement session in order to measure a specific physiological parameter. That is, a measurement session is initiated each time the selected measurement criterion or set of criterions is satisfied, and, accordingly, there is possible to obtain substantially repeatable measurements. For example, if the physiological parameter of interest is to be measured when the patient is resting the measurement criterion, or in other words the preferred signal combination, can be when a low body activity has been detected for a predetermined period, e.g. more than 5 minutes, and the position sensor indicates that the patient is in supine. According to another example, the specific physiological parameter to be measured is the intra thoracic impedance. In this case, the measurement criteria may be that the patient is in a rest mode and is lying on his or hers back. In addition there is a number of further measurement conditions that can be used, e.g. that the measurement session is only initiated during a specific period of the day, that the heart rate level is within a specific range, that a specified period of time has elapsed since the preceding measurement session, etc.

Of course, as the skilled man realizes, there a number of conceivable physiological parameters for which there is an interest of finding a suitable position for performing repeatable measurements, e.g. blood pressure (P), blood pressure variability (dP/dt) contractility, endocardial acceleration, blood flow, coronary blood flow, electrical bio-impedance, such as the cardiogenic impedance or the intra thoracic impedance, etc.

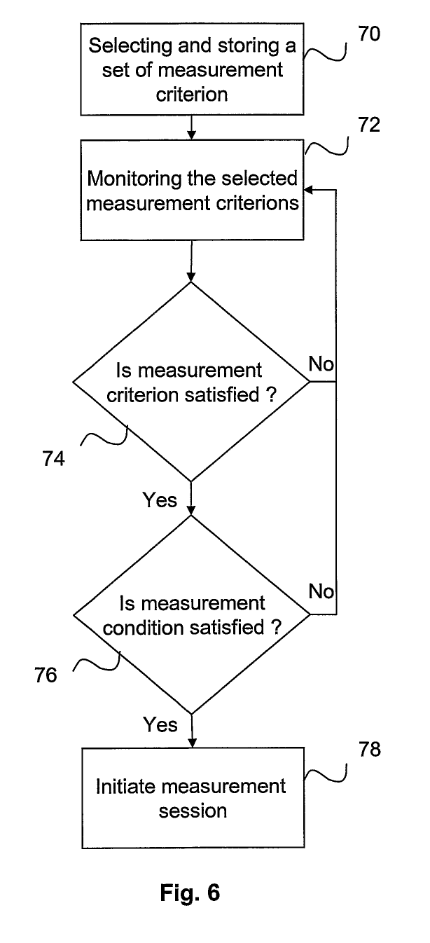

With reference now to FIG. 6, the procedure for performing measurements after the patient monitoring period has been completed according to one embodiment of the present invention will be discussed. This example is related to measurements of the electrical bio-impedance, or in fact the intra thoracic impedance, but, as discussed above, there are a number of other conceivable physiological parameters that can be measured. There are a number of possible impedance configurations, i.e. ways of injecting current between two electrodes in the pacemaker and then to measure the voltage the current provokes between the electrodes. For example, impedance configurations can be uni-polar, bi-polar, tri-polar or quadro-polar. The configuration denominated as bi-polar means, in practice, a configuration where the current and the voltage is sent out and measured between the same two electrodes. When one of the electrodes used in a bi-polar measurement is the housing or the case, the configuration is called uni-polar. For example, in FIG. 1, between the housing of the pacemaker 2 and a right ventricular electrode arranged at the distal end of lead 6a. A tri-polar configuration uses three electrodes, i.e. the current injection and the voltage measurement share one electrode. As an example, the current can be sent out from the housing or the case of the medical device to a RV-tip and the voltage is measured between the case and RV-ring. In quadro-polar measurements, the current is sent out between electrodes and the voltage is measured between two entirely different electrodes, i.e. in this case there are four electrodes involved.

First, at step 70, at least one measurement criterion is selected and memorized in the storing means 31 in accordance with the discussion above. In this example, the selected measurement criterion is when the patient is lying on his or hers back. The measurement criterion can be selected automatically by the medical device or can be manually programmed by means of the programmer, and it is also possible to, for example, change the criterion if necessary. At step 72, the specific parameters of the set of measurement criterion are monitored, i.e., in this case the position sensor 35 monitors or detects the position of the patient in order to detect when the patient is in the predetermined position, i.e. when lying on the back. During periods when the patient is in other positions than the predetermined specific position, the impedance sensing circuit 29 is in an idle mode. Then, at step 74, it is checked whether the predetermined measurement criterion(-s) is or are satisfied. If the predetermined measurement criterion is satisfied, i.e. the patient is in the predetermined body position, it is checked, in step 76, whether additional measurement conditions in addition to the predetermined measurement criterion is or are satisfied, if any selected. In this case it is checked whether a specified period of time has elapsed since the preceding measurement session, for example, the condition may be that the sensing circuit is refractory during 1 hour after a valid measurement session. Otherwise, the procedure returns to step 72. Of course, the procedural steps 74 and 76 can be performed in one step as an alternative. If is determined that the additional measurements condition(-s) is (are) satisfied, the device proceeds to step 78, where the microprocessor 30 sends a triggering signal to the control circuit 27, which, in turn, puts the impedance sensing circuit 29 in an active mode where the sensing circuit 29 initiates an impedance sensing session, which may be performed in accordance with conventional practice.

Although an exemplary embodiment of the present invention has been shown and described, it will be apparent to those having ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims.

We claim as our invention:

1. A method for evaluating the prevalence of different postures of a patient, comprising the steps of
sensing signals indicating the posture of said patient during a monitoring period having a predetermined length;
determining specific body postures of the patient during said monitoring period using said signals;
measuring the amount of time, the patient spends in each of said specific postures; storing information regarding each specific posture and the amount of time spent in each posture; and
evaluating the prevalence of said different postures of the patient by classifying said stored information with respect of specific postures and the amount of time spent in corresponding postures.

2. The method according to claim 1, further comprising the step of selecting at least one measurement criterion in dependence of said evaluation; and
storing said selected first measurement criterion.

3. The method according to claim 2, comprising employing a specific body posture of the patient as said at least one measurement criterion.

4. The method according to claim 2, further comprising the step of initiating a measurement session in order to measure at least one physiological parameter when said selected at least one measurement criterion is satisfied.

5. The method according to claim 1, further comprising the steps of:
sensing an activity level of said patient during said monitoring period;
determining whether said activity level is within at least one predetermined range storing information regarding the activity level range of the patient together with each specific posture and the amount of time spent in each posture; and
using said information regarding the activity level range of the patient in said evaluation by classifying said information with respect of specific postures, the amount of time spent in corresponding postures, and activity level range.

6. The method according to claim 5, further comprising the step of selecting a second measurement criterion of said at least one measurement criterion, in dependence of said evaluation, for measurement of said least one specific physiological parameter; and storing said selected second measurement criterion.

7. The method according to claim 6, comprising employing a specific activity level range of the patient as said second measurement criterion.

8. The method according to claim 1, wherein the step of evaluating comprises classifying said information as to distribution of said periods of time said patient spends in a specific posture over said monitoring period.

9. The method according to claim 1, further comprising initiating a measurement session if said selected at least one measurement criterion is satisfied during a predetermined period of time of the day.

10. The method according to claim 1, further comprising the steps of:
sensing a heart rate of the patient;
determining whether the heart rate is within a predetermined range; and
using said predetermined heart rate range as a measurement criterion.

11. The method according to claim 1, further comprising:
at the initiation of a measurement session, measuring an amount of time elapsed since a preceding measurement session; and
cancelling initiation of the measurement session if the amount of time elapsed since said preceding measurement session is within a predetermined range.

12. (canceled)

13. The method according to claim 1 comprising, in said sensor measurement session measuring a physiological characteristic selected from the group consisting of blood pressure, blood pressure variation, heart sound, contractility, endocardial acceleration, blood flow, coronary blood flow, electrical bio-impedance, cardiogenic impedance, and infra thoracic impedance.

14. A medical device for evaluating the prevalence of different postures of a patient comprising
a position sensor that senses the posture of said patient;
a determining unit that determines specific body postures of the patient during a monitoring period having a predetermined length using position signals from said position sensor;
a timer that measures an amount of time the patient spends in each of said specific postures;
a storage unit that stores information regarding each specific posture and the amount of time spent in each posture; and
an evaluation unit that evaluates the prevalence of said different postures of the patient by classifying said stored information with respect of specific postures and the amount of time spent in corresponding postures.

15. The medical device according to claim 14, wherein the storage unit stores at least one selected measurement criterion, said measurement criterion being selected in dependence on said evaluation.

16. The medical device according to claim 15, wherein said at least one measurement criterion is a specific body posture of the patient.

17. The medical device according to claim 15, further comprising:
a measurement circuit that executes a measurement session in order to measure a physiological parameter of said patient;
a measurement criterion determining unit connected to said position sensor and said measurement circuit, that determines whether said at least one measurement criterion is satisfied; and
if said at least one measurement criterion is satisfied, said measurement criterion determining unit applying a triggering signal to said measurement circuit, wherein said measurement circuit, upon receiving said triggering signal, initiates a measurement session.

18. The medical device according to claim 14, further comprising:
an activity level sensor or that senses an activity level of said patient;
an activity level determining unit that determines whether said sensed activity level is within at least one predetermined range;
said storage unit storing information regarding the activity level range of the patient together with each specific posture and the amount of time spent in each posture; and
said evaluation unit the prevalence of said different postures of the patient by classifying said stored information with respect of specific postures, the amount of time spent in corresponding postures, and the activity level range.

19. The medical device according to claim 18, wherein said storage unit stores a second measurement criterion of said at least one measurement criterion, said second measurement criterion being selected in dependence on said evaluation.

20. The medical device according to claim 19, wherein said second measurement criterion is a specific activity level range of the patient.

21. The medical device according to claim 14, wherein said evaluation unit classifies said information as to distribution of periods of time said patient spends in a specific posture over said predetermined patient monitoring period.

22. The medical device according to claim 16, wherein said measurement criterion determining unit applies said triggering signal to said measurement circuit if said selected at least one measurement criterion is satisfied during a predetermined period of time of the day.

23. The medical device according to claim 14, further comprising:
a rate sensor that senses a heart rate of the patient;
means for determining whether the heart rate is within a predetermined range; and
wherein said storing means is arranged to store said predetermined heart rate range as a measurement criterion.

24. The medical device according to claim 16, wherein:
said timer, at the initiation of a measurement session, measures the amount of time elapsed since the preceding measurement session; and applies a signal to said measurement circuit cancelling the initiation of the measurement session if the amount of time elapsed since said preceding measurement session is within a predetermined range.

25-28. (canceled)

29. The medical device according to claim 14, wherein said measurement unit senses any physiological characteristic selected from the group consisting of blood pressure, blood pressure variation, heart sound, contractility, endocardial acceleration, blood flow, coronary blood flow, bio-impedance, intra-thoracic and cardiogenic impedance.

30. (canceled)

31. A computer-readable medium encoded with a data structure, for use with a computer connected to a sensor, said data structure, when said computer-readable medium is loaded in the computer, causing the computer 2:
sense signals with said sensor indicating the posture of a patient during a monitoring period having a predetermined length;
determine specific body postures of the patient during said monitoring period using said signals;
measure the amount of time, the patient spends in each of said specific postures; storing information regarding each specific posture and the amount of time spent in each posture; and
evaluate the prevalence of said different postures of the patient by classifying said stored information with respect of specific postures and the amount of time spent in corresponding postures.
